(19) United States
(12) Patent Application Publication
(10) Pub. No.: US 2008/0292586 A1
(43) Pub. Date: Nov. 27, 2008

(54) Method and Use of Interferon Compositions For the Treatment of Avian Influenza

(76) Inventors: Karen Jervis, Midlothian (GB); Paula Barnard, Midlothian (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/883,588

(22) PCT Filed: Feb. 6, 2006
(86) PCT No.: PCT/GB2006/000400

§ 371 Date: May 29, 2008

Related U.S. Application Data
(60) Provisional application No. 60/658,065, filed on Mar. 3, 2005.

(30) Foreign Application Priority Data
Feb. 4, 2005 (GB) 0502341.1
May 3, 2005 (GB) 0508963.6

(51) Int. Cl. A61K 38/21; A61P 31/12

(52) U.S. Cl. 424/85.6; 424/85.4; 424/85.7

(57) ABSTRACT

The present invention provides methods and uses for an interferon composition in the treatment of avian influenza. Transmission of avian influenza virus H5N1 to humans has been shown to be highly pathogenic. The present invention provides for methods of treatment that provide a broad spectrum, first line defense against avian influenza infection in humans. The methods of the present invention can be further extended to treat strains of avian influenza viruses that have resulted from antigenic drift, this potentially resulting in avian influenza based virus which is highly pathogenic and transmissible between humans.

### FIELD OF THE INVENTION

The present invention provides a composition for use in the treatment of an avian orthomyxovirus infection in humans, more specifically a type A influenza virus of the family orthomyxoviridae infection, most specifically Influenza virus A, subtypes H5 (including H5N1), H7 and H9 (commonly termed “avian influenza” or “bird flu”).

### BACKGROUND TO THE INVENTION

Avian influenza H5N1, the strain of influenza commonly known as bird flu, was first isolated from birds in South Africa in 1961. Wild birds are the natural host of the virus, with the virus circulating amongst birds worldwide. Avian influenza H5N1 is extremely contagious and can be deadly to domesticated poultry. H5N1 is one of fifteen subtypes of influenza virus are known to infect avians, however, there have been previous instances of certain subtypes of avian influenza strains “jumping” the species barrier and causing infection in humans. Most recently, avian influenza A virus subtypes H5 (H5N1), H7 and H9 have been found to cause infection in humans.

Since January 2003, outbreaks of H5N1 have caused incidences of avian and human infection in several countries around the world. Infection with this H5N1 subtype was first detected in Thailand and Vietnam; from Dec. 30, 2003, to Mar. 17, 2004, 12 confirmed human cases of avian influenza H5N1 were reported in Thailand and 23 in Vietnam, resulting in a total of 23 deaths. Further, since January 2004, 161 humans have been infected with H5N1 in Vietnam, Thailand, Cambodia, China, Indonesia, and recently, Turkey and Iraq. 86 of these cases have been fatal (source: WHO).

Infections in humans coincided with devastating epidemics in poultry farms in Asian countries, with a reported mortality rate approaching 100%. It is now acknowledged that H5N1 influenza virus is endemic in Asian domestic fowl, and unlikely to be eradicated (reviewed in Moscona, 2004; http://www.who.int/csr/disease/influenza/H5N1-9reduit.pdf). Most recently, H5N1 influenza has been noted to have spread from South East Asia through Russia (Siberia), Kazakhstan, Romania and Turkey.

It is clear that the cultural practice of keeping animals in close proximity to each other as well as with humans has been the source of cross-species infection. To control any outbreak, it is necessary for thousands of chickens to be slaughtered to remove the source of the virus. Where transmission into humans was observed, spread of the virus was primarily from bird to human, with only rare person-to-person spread noted (Shortridge et al, 1998).

### Virus Reassortment and Transmission

Influenza viruses are orthomyxoviruses, and fall into three types; A, B and C. Influenza A and B virus particles contain a genome of negative sense, single-stranded RNA divided into 8 linear segments. Co-infection of a single host with two different influenza viruses may result in the generation of ‘reassortant’ progeny viruses having a new combination of genome segments, derived from each of the parental viruses (reviewed in Baigent and McCauley, 2003).

Influenza A viruses have been responsible for four recent pandemics of severe human respiratory illness. Influenza A viruses can be divided into subtypes according to their surface proteins, hemagglutinin (HA or H) and neuraminidase (NA or N). There are 14 known H subtypes and 9 known N subtypes. All H subtypes have been found in birds, however only three H subtypes (H1, H2 and H3) and two N subtypes (N1 and N2) have been reported as commonly circulating in humans (reviewed in Baigent and McCauley, 2003).

Seasonal influenza epidemics in humans are associated with amino acid changes in antigenic sites in the hemagglutinin and neuraminidase proteins, in a process termed ‘antigenic drift’. Major pandemics are associated with the introduction of new hemagglutinin and neuraminidase genes from animal-derived influenza viruses, by reassortment, into the genetic background of a currently circulating human virus—called ‘antigenic shift’.

H5N1 isolates from geese, ducks, and chickens from farms and poultry markets in Hong Kong during the 1997 outbreak were compared with a human isolate and demonstrated to replicate in geese, pigs, rats and mice (Shortridge et al, 1998). Animal to animal transfer was not observed for mice or pigs. As pigs are receptive to avian, human and swine influenza types, they have long been thought of as a potential “mixing vessel” for antigenic shift to occur, allowing the virus to acquire human influenza-type genes permitting human to human transmission (Baigent and McCauley, 2003). However, more recent outbreaks have provided a clear indication that some avian influenza viruses have the potential to directly infect humans without a swine intermediate as a mixing vessel (Suarez et al, 1998).

H5N1 mutates rapidly and has a documented propensity to acquire genes from influenza viruses infecting other animal species. Its ability to cause severe disease in humans has now been documented on multiple occasions. In addition, laboratory studies have demonstrated that isolates from this virus have a high pathogenicity in vitro and in vivo. Birds that survive infection excrete virus for at least 10 days, orally and in faeces, thus facilitating further spread at live poultry markets and farms, and by migratory birds.

The influenza pandemic of 1918-1919, when a new influenza virus emerged and rapidly spread around the globe, killed an estimated 40-50 million people in a period of two years (Taubenberger et al, 2000). Accordingly, the importance of establishing a reliable prophylactic and/or therapeutic treatment for not only sporadic outbreaks of H5N1 and other avian influenza virus infections in humans, but also for use in the event that a pandemic situation arises, is clearly evident.

### Transmission of H5N1 Influenza Virus Infection to the Human Host

It is not fully known how or why H5N1 has crossed the species barrier, causing infection in humans. Since the natural host for Influenza A viruses are free-living aquatic birds of the orders Charadriiformes and Anseriformes (Easterday et al, 1997; Kawaoka et al, 1988; Slemons et al, 1974), infection of domestic poultry (chicken, geese, turkeys) indicates a cross-species jump has already occurred (Suarez et al, 1998).

It is clear that upon crossing the species barrier, pathogenicity of H5N1 is high. The H5N1 virus comes in two forms, one demonstrating low pathogenicity in chickens, and the second being the highly virulent form known as “highly pathogenic avian influenza”. There is mounting evidence that this strain has the capacity to jump the species barrier causing severe disease, with high mortality, observed in humans.

The direct infection of the H5N1 avian influenza virus into humans presents a high risk potential for progression to pandemic spread amongst humans. Repeated chances at replication in humans may allow this virus to become better adapted to humans and allow efficient human-to-human transmission (Suarez et al, 1998). Indeed, co-infection of a host with both Avian influenza virus and human influenza virus may result in reassortment and emergence of an influenza virus with the high pathogenic characteristics of the avian virus, and human-to-human transmissibility similar to a human influenza virus, as well as viral factors not previously seen by a naïve human population.

### Current Therapies

At present, there is no vaccine against H5N1 for use in humans. According to the WHO, vaccines in development against the 2003 strain of H5N1 are not protective against the 2004 Vietnam H5N1 strain, which early studies suggest has mutated (due to antigenic drift) significantly. Vaccine development will not be possible until the human-to-human transmissible strain emerges, and will then take a number of months to be ready for wide scale administration.

It is clear that preventing a pandemic by way of vaccination is not a reliable means of control of influenza, largely due to the short time period between strain detection and need for the product (Hilleman, 2002). Therefore, the development of a broad-spectrum means to control influenza infection, in the form of safe and effective anti-viral therapy would be highly desirable.

At present, there are two classes of drugs commercially available for the prevention and treatment of influenza virus infections in humans; M2 ion channel blockers and Neuraminidase inhibitors.

Amantadine and Rimantadine function by blocking the ion channel activity of the viral M2 protein (Hilleman, 2002; Ludwig et al, 2003), which is mainly required during virus entry in the early phase of the replication life cycle. Both treatments are highly effective in treating influenza A but cause significant side effects on the central nervous system, liver and kidneys. Sensitive influenza strains rapidly develop resistance in vitro and in vivo (Fleming, 2001). Initial analyses on H5N1 virus isolates from Vietnam viruses are resistant to the M2 inhibitors (Available at URL www.who.int/csr/disease/avian_influenza/avian_faqs/e n/print.html). M2 inhibitor-resistant influenza viruses are generated in up to 30% of patients, and these viruses are virulent and transmissible (Moscona, 2004).

Oseltamivir and Zanamivir block the action of neuraminidase to prevent the release of newly formed virus from infected cells and spread within the host (Hilleman, 2002; Ludwig et al, 2000). Both drugs efficiently inhibited non-avian derived influenza viruses in clinical studies (Dreitlein et al, 2001), however escape from the selective pressures of neuraminidase inhibitors has been observed in cell culture and in patients (Gubareva et al, 1998; Gubareva et al, 2002).

Oseltamivir phosphate (Tamiflu from Roche) is currently the only antiviral treatment proposed for treatment of the H5N1 Avian influenza in humans.

Many of the current anti-viral therapies are directed towards targeting viral components and are therefore prone to compensatory viral escape mechanisms (Ludwig et al, 2003). For example, studies using a genetically manipulated influenza virus (NWS/G70C) containing the neuraminidase gene from an Avian influenza virus (A/tern/Australia/G70C/75) was demonstrated to acquire resistance to all neuraminidase inhibitors tested, including Oseltamivir carboxylate (GS4071) (McKimm-Breschkin et al., 1998), the active compound of the ethyl ester prodrug Oseltamivir phosphate, which is the basis for Roche's Tamiflu.

To date, Oseltamivir-resistant influenza viruses have been less transmissible or pathogenic, however the high frequency of emergent, resistant viruses indicates that it is only a matter of time before a resistant, highly transmissible virus emerges, and this raises concerns about the widespread use of Oseltamivir in a pandemic situation (Moscona, 2004). A recent report (de Jong et al., 2005 NEJM 353 2667-72) suggested that two Vietnamese patients treated with Tamiflu were resistant to the drug and died from avian influenza. This again indicates the need for alternative therapy options.

Treatments aimed at manipulating the host to interfere with viral replication, either by enhancing antiviral responses or by inhibiting proviral activities within the host cell have greater potential to control influenza without selective pressure on the virus itself to mutate in a compensatory manner are desirable. The possibility for combination therapy targeting virus and host at the same time, to minimise the opportunity for the virus to acquire resistance is also particularly appealing.

Ribavirin™ is a broad-spectrum anti-viral agent based on a purine nucleoside analogue and is the standard treatment regimen for hepatitis C.

Ribavirin is known to be active against various RNA viruses by inducing lethal mutagenesis of the viral RNA genome (Crotty et al., 2000, Tam et al., 2001) and is known to show anti-viral activity against animal coronaviruses (Weiss & Oostrom-Ram, 1989, Sidwell et al., 1987). Although Ribavirin has a marked anti-viral activity against a number of viruses, it is not acknowledged as a medicament for influenza infections. In addition, the considerable toxicity associated with Ribavirin limits its utility as a medicament.

### Anti-Viral Effects of Interferon

The family of proteins known as the interferons (IFNs) can elicit a powerful anti-viral response, in addition to being pleiotropic effectors of the immune system (for a review, see Stewart, 1979). The interferons may be classified into two groups—Type I interferons and Type II interferons. The type I interferons consist of interferon Alpha and interferon Beta, whereas the Type II group consists of interferon Gamma. Type I interferons are produced in direct response to a viral infection.

Interferon Alpha is represented by a large family of structurally related genes expressing at least thirteen subtypes, whereas interferon Beta is encoded by a single gene (Diaz et al., 1996). Both types of interferon are able to stimulate an ‘anti-viral’ state in target cells, whereby the replication of a virus is inhibited through the synthesis of enzymes which interfere with the cellular and viral processes. Type I interferons also act to inhibit or slow the growth of target cells and may render them more susceptible to apoptosis. This has the effect of limiting the extent of viral spread. Type I interferons are immunomodulators, or ‘biological response modifiers’, which act to stimulate the immune response. Even though IFN Alpha and IFN Beta show many broad similarities in their actions, there are significant differences in the manner by which they exert their effects and it is these extended functions that account for the different ranges of antiviral activities of the two types.

The interferon response is an extremely efficient anti-viral mechanism and the effectiveness of this response has exerted pressure on viruses to evolve means to circumvent the interferon's anti-viral effect. The anti-viral response of interferon Alpha is not a virus-specific response and has the potential of being used to counteract infections of a very broad range of viruses. Indeed, interferon Alpha is the ‘first line of defense’ against viral infection—the expression of interferon Alpha occurs as a very early response to infection and precedes the majority of the other innate-immune response cytokines, to induce a ‘priming’ state against the infection (Biron, 1998). Interferon Alpha also shows a synergistic effect with other early response cytokines such as transforming growth factor alpha (TGF Alpha), which has led to the suggestion by many researchers that interferon Alpha is the first and most important cytokine produced by the antigen presenting cells (APC) following infection (Biron, 1998).

### Interferon Alpha as a Possible Treatment for Influenza

A double-blind, controlled trial on the preventative effect of human interferon Alpha on upper respiratory viral infections carried out during the period of outbreaks of influenza type A epidemics demonstrated that clinical manifestations referable to influenza virus infection were milder in the interferon-treated group than in the control group (Isomura et al, 1982). However, there was no significant difference in the serological responses of the two groups after infection with Influenza virus type A. Human leukocyte-derived interferon demonstrated a prophylactic effect on upper respiratory disease, where alleviation of subjective symptoms and temperature reductions were reported (Imanishi, et al, 1980). In contrast, the efficacy of recombinant interferon Alpha 2A in the prophylaxis of acute upper respiratory tract infection during a period that coincided with an outbreak of H3N2 influenza failed to demonstrate clinical benefit from the use of interferon Alpha in the amelioration of symptoms (Tannock et al, 1988).

Influenza patients secrete interferons and other cytokines in the host (Hayden et al., 1998). This is despite influenza viruses possessing a mechanism to inhibit dsRNA induction of type I interferon expression and, to some degree, responses to interferon (reviewed in Krug et al, 2003). The major in vivo function of the non-structural-protein-1 (NS1) appears to be to antagonise the anti-viral type I interferon system. NS1 appears to achieve this by binding and sequestering dsRNA which would otherwise activate components of the anti-viral response, including interferon Regulatory Factor-3 (IRF3), NF-κB, Jun N-terminal kinase and its AP-1 transcription factor substrates and the dsRNA-dependent kinase, PKR (reviewed in Baigent and McCauley, 2003). These are all implemented primarily in the induction of type I IFN expression; however, PKR is also active in anti-viral and anti-proliferative responses to interferon Alpha (Goodbourn et al, 2000). Despite this function of NS1, the majority of influenza viruses remain sensitive to IFN Alpha treatment in vitro (reviewed in Baigent and McCauley, 2003). The IFN-inducible Mx proteins possess anti-influenza virus activity. Therefore it would seem that the major role of NS1 is in preventing induction of type I IFN rather than response to IFN Alpha.

In contrast, avian influenza H5N1 virus isolates from the Hong Kong outbreak of 1997 have been demonstrated to be resistant to anti-viral effects induced by recombinant swine IFN Alpha in swine in vitro and in vivo models, where other influenza subtypes were found to be sensitive (Seo et al, 2002; Seo et al, 2004). Production of recombinant H1N1-type influenza virus particles containing the H5N1 NS1 gene demonstrated that NS1 was essential in developing this resistance, that possession of a glutamic acid at residue 92 in NS1 was required, and that this residue might confer resistance to degradation within the host cell. This highlights the unusual nature of the H5N1 influenza virus.

Levels of interleukin 1 alpha, 1 beta, and 6, interferon gamma, and chemokine CXCL1 were found to be high in extracts of pulmonary tissue of mice infected with genetically manipulated influenza virus containing the H5N1 NS1 gene (Lipatov et al., 2005), which has been linked with the high pathogenesis of H5N1 influenza. Exceptionally high levels of proinflammatory cytokines, interferon beta, and, most notably, TNF-alpha were found in two humans who died of H5N1/1997 infection (Cheung et al, 2002). Full post-mortem reports described reactive hemophagocytic syndrome with elevated concentrations of interleukin 6, TNF-alpha, and interferon gamma (To et al., 2001).

### Multi-Subtype IFN Alpha as a Treatment for Avian Influenza H5N1

In the previously described study carried out in a swine model, both in vitro and in vivo, the type I IFN used was a recombinant IFN Alpha produced in an Escherichia coli expression system. Recombinant IFNs, which consist of only the IFN Alpha 2 subtype, currently dominate the market for anti-viral and oncology indications. The two main commercially available recombinant alpha IFN products are Intron A™ from Schering Plough (IFN-Alpha 2b) and Roferon™ (IFN-Alpha 2a) from Roche. These two allelic variants of the alpha 2 subtype differ by only one amino acid residue. PEGylated versions of these interferon products are now in clinical use and reportedly demonstrate improved pharmacokinetics in vivo when compared to their non-PEGylated versions.

In contrast to these single-subtype recombinant products, there are several alpha IFN preparations that consist of a mixture of different subtypes, so-called multi-subtype compositions. These multi-subtype IFN Alpha products are produced either by human leukocytes in response to stimulation by a virus (examples of products produced in this way are Multiferon™: Viragen, or Alferon-N: Hemispherx), or are produced in human lymphoblastoid cells, cultured from a patient with Burkitt's lymphoma (such as Sumiferon: Sumitomo).

There are many differences between the recombinant forms of IFN Alpha and the multi-subtype forms. The most obvious difference is the number of IFN Alpha subtypes each possesses. The multi-subtype forms of IFN Alpha comprise many subtypes of IFN Alpha. The recombinant Interferon Alpha 2 produced by human cells in the manufacturing process of the multi-subtype forms is glycosylated, whereas the recombinant forms are unglycosylated due to their production in bacterial systems. Glycosylation plays a major role in many functions of the protein product, such as half-life, the bioactivity and its immunogenicity. Therefore, the glycosylation of a product is an important consideration when developing a therapeutic or prophylactic treatment, as it may affect the duration in the body after administration, the activity of a therapeutically appropriate dose and the tolerability to the product itself.

There is much debate over the importance of the various subtypes of IFN Alpha. It has been suggested that the different subtypes have different roles in vivo. Studies using mammalian cells have demonstrated that differences do in fact exist between the anti-viral activities of the various subtypes of IFN Alpha (Weck et al., 1981). Studies to examine the effects of highly purified subtypes produced by recombinant DNA technology appeared to show only modest variations in their relative anti-viral effects (Allan & Fantes, 1980; Goeddel et al., 1981), whereas studies on the subtypes obtained from purifying the ‘natural’ mixtures of subtypes produced using human buffy coats demonstrate a clear and significant variation between the different subtypes in terms of their anti-viral effect in human cell lines (Foster et al., 1996; Fish et al., 1983). Foster et al., 1996 demonstrated that the leukocyte-derived IFN Alpha 8 subtype had the most potent anti-viral effect of all the subtypes tested. The authors postulated that post-translational modification of this subtype may play a significant role in determining its activity. The fact that synthetic mixtures of the subtypes did not have as potent an anti-viral effect as the leukocyte derived mixtures suggests that the different subtypes, as produced naturally by the cell, act in a synergistic manner with each other, in a manner which is not as yet understood.

Through enhancement of the immunological effector functions, it can be seen that IFN Alpha imparts a potent anti-viral defense mechanism that has been demonstrated to have a cytopathic effect against a wide range of pathogens. The multi-subtype form of IFN Alpha has characteristics that may present certain advantages when used as a medicament to treat or prevent such a virus.

There are currently no completely effective therapeutic or prophylactic treatments for humans infected with Influenza A virus subtype H5N1. The potential severity and mortality linked to human infection with Influenza A (H5N1), other H5 strains and other subtypes such as H7 and H9 illustrates that there exists a need for an effective treatment. Further, the potential for reassortment of the virus into a form which is transmissible from human to human identifies the requirement for a specific therapy which can be administered widely following the identification of such a reasserted viral variant.

### SUMMARY OF THE INVENTION

The present inventors have surprisingly shown that interferons, and in particular multiple subtype natural human alpha interferon products are surprisingly effective for the treatment and prophylaxis of type A Influenza virus infection in humans which has been derived from avian influenza.

According to a first aspect of the present invention there is provided a method for the treatment or prophylaxis of human infection with type A Influenza characterised in that the virus has a hemagglutinin component of subtype H5, H7 or H9, the method comprising the steps of:
providing a composition comprising a type I interferon, and
administering a therapeutically useful amount of said composition to a subject in need of treatment.

In specific embodiments, the subtype of the type A Influenza virus may be further defined as being of the strain H5N1, H9N2, H7N2, H7N3 or H7N7.

In further specific embodiments, the interferon may be any suitable type I interferon, for example interferon alpha or interferon beta, but is preferably interferon alpha. Where the interferon is interferon alpha, the interferon composition of the invention may be comprised of a single subtype or alternatively, in a further embodiment of a plurality of subtypes of interferon alpha.

It is preferred that the interferon be glycosylated, and accordingly, where a recombinant form of interferon is used, it is preferred that the recombinant production system allows for glycosylation.

Most preferably however, the interferon is naturally derived. In one embodiment, the naturally derived interferon is obtained from leukocytes following viral stimulation. For example, the interferon may be produced in human lymphoblastoid cells cultured from a patient with Burkitt's lymphoma.

In one embodiment, the preferred interferon compositions for use in the present invention includes multi-subtype compositions comprising at least two interferon alpha (IFN-α) subtypes. In particular a multi-subtype interferon alpha composition comprising a mixture of the subtypes; alpha 1, alpha 2, alpha 8, alpha 10, alpha 14 and alpha 21 is preferred. In various further embodiments, the interferon composition may comprise other interferon alpha subtypes, interferon αn1, interferon αn3 or interferon β1 a or b.

In a further preferred embodiment of the invention, the interferon composition is the multi-subtype naturally derived interferon alpha product commercially available from Viragen, Inc. or any of its subsidiaries under the trade name Multiferon™. Multiferon™ is a highly purified natural multi-subtype human alpha interferon product derived from human white blood cells. Multiferon™ may be produced in accordance with the teachings set forth in International PCT Patent Publications No WO 00/39163 or WO 90/15817. The Multiferon™ composition comprises interferon alpha subtypes alpha 1, alpha 2, alpha 8, alpha 10, alpha 14 and alpha 21. Multiferon™ contains interferon alpha subtypes of the following proportions by weight: alpha 1 at 37+/−9%, alpha 2 and alpha 21 at 30+/−7%, alpha 8 plus alpha 10 at 22+/−6%, and alpha 14 at 11+/−3%.

Accordingly a further embodiment of this aspect of the invention provides for an interferon composition which comprises at least interferon alphas of the subtypes 1, 2, 8, 10, 14 and 21. In further specific embodiments at least one further interferon alpha subtype may be added to this composition. Similarly, at least one interferon alpha subtype may be removed from this composition. In a yet further specific embodiment, the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

According to a second aspect of the present invention there is provided the use of a type I interferon composition in the treatment or prophylaxis of human infection with a type A Influenza subtype defined by the presence of the hemagglutinin subtype H5, H7 or H9.

In further embodiments, the type A Influenza subtype is of the strain H5N1, H9N2, H7N2, H7N3 or H7N7.

In yet further embodiments the type A influenza subtype may comprise hemagglutinin of subtype H5, H7 or H9 along with any neuraminidase subtype.

The interferon composition may be any suitable interferon, as defined in relation to the first aspect of the invention. Specifically, the interferon composition may comprise interferon alpha of the subtypes 1, 2, 8, 10, 14 and 21. In further specific embodiments at least one further interferon alpha subtype may be added to this composition. Similarly, at least one interferon alpha subtype may be removed from this composition. In a yet further specific embodiment, the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

According to a third aspect of the present invention there is provided the use of interferon in the preparation of a medicament for the treatment or prevention of infection with a type A avian influenza virus.

In one embodiment, the type A avian influenza virus may be defined by the presence of the hemagglutinin subtype H5, H7 or H9.

In further embodiments, the type A Influenza virus is of the strain H5N1, H9N2, H7N2, H7N3 or H7N7.

In yet further embodiment the type A influenza virus may comprise hemagglutinin of subtype H5, H7 or H9 along with any neuraminidase subtype.

The interferon composition may be any suitable interferon, as defined in relation to the first aspect of the invention. Specifically, the interferon composition may comprise interferon alpha of the subtypes 1, 2, 8, 10, 14 and 21. In further specific embodiments at least one further interferon alpha subtype may be added to this composition. Similarly, at least one interferon alpha subtype may be removed from this composition. In a yet further specific embodiment, the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

According to a yet further aspect of the present invention there is provided an interferon composition comprising at least interferon alpha subtypes 1, 2, 8, 10, 14 and 21 for use in the treatment of an avian influenza infection in humans.

In one preferred embodiment, the interferon composition comprises interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

According to a further aspect of the present invention there is provided a pharmaceutical composition for the treatment of an avian influenza infection, wherein said composition comprises interferon along with a pharmaceutically acceptable excipient, carrier or diluent.

The present inventors have also identified that the interferon composition of the present invention may be administered along with a second anti-viral composition. This would provide a combination therapy which may have utility in relation to a viral infection which has a particularly high pathogenicity.

Accordingly, a further aspect of the present invention provides a method for preventing or treating human infection with avian influenza, the method comprising the steps of;
providing a composition comprising a type I interferon,
administering a therapeutically useful amount of said composition to a subject in need of treatment, and
further administering a therapeutically useful amount of a suitable secondary anti-viral compound.

In one embodiment, the anti-viral compound is administered along with the interferon composition, however, in further embodiments, the secondary anti-viral compound may be administered before or after the interferon composition has been administered.

In one embodiment, the type A avian influenza virus may be defined by the presence of the hemagglutinin subtype H5, H7 or H9.

In further embodiments, the type A Influenza virus is of the strain H5N1, H9N2, H7N2, H7N3 or H7N7.

In yet further embodiments the type A influenza virus may comprise hemagglutinin of subtype H5, H7 or H9 along with any neuraminidase subtype.

The interferon composition may be any suitable interferon, as defined in relation to the first aspect of the invention. Specifically, the interferon composition may comprise interferon alpha of the subtypes 1, 2, 8, 10, 14 and 21. In further specific embodiments at least one further interferon alpha subtype may be added to this composition. Similarly, at least one interferon alpha subtype may be removed from this composition. In a yet further specific embodiment, the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

In preferred embodiments, the secondary anti-viral compound may be selected from the group comprising; ribavirin, amantadine, rimantadine, oseltamivir (Tamiflu™) or zanamivir.

According to a yet further aspect of the present invention there is provided the use of interferon and an anti-viral compound in the preparation of a combined medicament for the treatment or prevention of infection with type A Influenza subtype H5, H7 or H9.

In one embodiment the type A Influenza subtype is of the strain H5N1, H9N2, H7N2, H7N3 or H7N7.

In preferred embodiment, the secondary anti-viral compound may be selected from the group comprising; ribavirin, amantadine, rimantadine, oseltamivir (Tamiflu™) or zanamivir.

The interferon composition may be any suitable interferon, as defined in relation to the first aspect of the invention. Specifically, the interferon composition may comprise interferon alpha of the subtypes 1, 2, 8, 10, 14 and 21. In further specific embodiments at least one further interferon alpha subtype may be added to this composition. Similarly, at least one interferon alpha subtype may be removed from this composition. In a yet further specific embodiment, the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

### Reassortment of Further Avian Influenza Strains

There are 3 prominent subtypes of avian influenza virus; H5, H7 and H9. Each of these 3 viral subtypes can potentially be combined with any one of the 9 neuraminidase surface proteins, hence there is the potential for up to 9 different forms of each subtype, for example H7N1, H7N2 up to H7N9.

As such, a further aspect of the present invention provides for the use of a type I interferon composition in the preparation of a medicament for the prevention and treatment of human infection with type A Influenza subtype H5, H7 or H9 when each and any of the foregoing subtypes is combined with any one of the known neuraminidase surface proteins to form a specific strain of avian influenza virus.

As herein defined, the term “type I interferon” comprises all subtypes of interferon alpha and interferon beta. Interferon beta is found in 2 subtypes, 1a and 1b.

In one embodiment the interferon alpha composition is multi-subtype interferon alpha preparation.

The interferon composition may be any suitable interferon, as defined in relation to the first aspect of the invention. Specifically, the interferon composition may comprise interferon alpha of the subtypes 1, 2, 8, 10, 14 and 21. In further specific embodiments at least one further interferon alpha subtype may be added to this composition. Similarly, at least one interferon alpha subtype may be removed from this composition. In a yet further specific embodiment, the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

### Reassortment and New Influenza Subtype Formation

Influenza A viruses are found in many different animals, including ducks, chickens, pigs, whales, horses, and seals. However, certain subtypes of influenza A virus are specific to certain species, except for birds which are hosts to all subtypes of influenza A.

Influenza A viruses normally seen in one species can cross over and cause illness in another species. For example, H5N1 avian influenza was responsible for a recent outbreak of bird flu in the human population, while H7N7, H9N2 and H7N2 subtypes have also been associated with transmission over the species barrier and resultant infection in humans.

Avian influenza viruses may be transmitted to humans in two main ways; (i) directly from infected birds or from material contaminated with avian influenza virus, (ii) through an intermediate host, such as a pig.

Influenza viruses have eight separate gene segments. The segmented genome allows viruses from different species to mix and create a new influenza A virus if viruses from two different species infect the same person or animal. For example, if a pig were infected with a human influenza virus and an avian influenza virus at the same time, the viruses could reassort and produce a new virus that had most of the genes from the human virus, but a hemagglutinin and/or neuraminidase from the avian virus. The resulting new virus might then be able to infect humans and spread from person to person, but it would have surface proteins (hemagglutinin and/or neuraminidase) not previously seen in influenza viruses that infect humans.

This type of major change in the influenza A viruses is known as antigenic shift. Antigenic shift results when a new influenza A subtype to which most people have little or no immune protection infects humans. If this new virus causes illness in people and can be transmitted easily from person to person, an influenza pandemic can occur.

It also is possible that the process of reassortment could occur in a human who was infected with avian influenza and a human strain of influenza. Virus reassortment could create a new virus with hemagglutinin from the avian virus and other genes from the human virus. Theoretically, influenza A viruses with a hemagglutinin against which humans has little or no immunity that have reassorted with a human influenza virus are more likely to result in sustained human-to-human transmission and pandemic influenza.

Infection with type A influenza virus in humans is generally caused by subtypes comprising H1, H2 and H3 hemagglutinin subtypes which are combined with one of either the N1 or N2 neuraminidase subtypes.

Type A influenza virus which is derived from, and primarily infectious to avians, but which has crossed the species barrier to cause infection in humans has been observed for type A influenza virus with hemagglutinin subtypes H5, H7 and H9. These strains, such as H5N1, H7N2, H7N3 and H9N2 comprise avian H and N subtypes.

Reassortment of viruses in a host co-infected with both an avian type A influenza virus and a human type A influenza virus may result in a virus wherein an H or N component from a ‘human adapted’ type A influenza virus reassorts with an avian influenza virus.

The inventors have identified that due to the ability of the interferon compositions of the present invention to be effective against avian influenza variants which have resulted from both antigenic drift and antigenic shift, the interferon compositions of the present invention have be likely to have efficacy against new strains of type A influenza virus irrespective of what antigenic shift mutations to the viral genome occur.

Accordingly it is a further aspect of the present invention to provide for the use of a type I interferon composition in the preparation of a medicament for the prevention or treatment of human infection with type A Influenza subtype which has resulted from natural reassortment of influenza variants.

In particular, this aspect of the present invention extends to the use of type I interferon compositions in the preparation of a medicament for the prevention and treatment of an influenza subtype which has resulted from natural reassortment of human influenza virus with avian influenza virus to form a new influenza virus variant.

In one embodiment, the influenza virus which has resulted from re-assortment may contain an avian hemagglutinin subtype and a ‘human adapted’ neuraminidase subtype; or alternatively a ‘human adapted’ hemagglutinin subtype and an avian neuraminidase subtype. In one specific embodiment, the virus subtype may be H5N1 wherein the neuraminidase subtype is derived from an avian type A influenza virus and the hemagglutinin component is derived from a ‘human adapted’ type A influenza virus.

The interferon composition may be any suitable interferon, as defined in relation to the first aspect of the invention. Specifically, the interferon composition may comprise interferon alpha of the subtypes 1, 2, 8, 10, 14 and 21. In further specific embodiments at least one further interferon alpha subtype may be added to this composition. Similarly, at least one interferon alpha subtype may be removed from this composition. In a yet further specific embodiment, the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

A yet further aspect of the present invention provides a method of treating or preventing human infection with type A Influenza subtype which has resulted from natural reassortment of influenza variants, the method including the step of administering a therapeutically useful amount of an interferon to a subject in need of treatment.

The term ‘treatment’ is used herein to refer to any regime that can benefit a human or non-human animal. The treatment may be in respect of an existing condition or may be prophylactic (preventative treatment). Treatment may include curative, alleviation or prophylactic effects.

### Administration

Interferons of and for use in the present invention may be administered alone but will preferably be administered as a pharmaceutical composition, which will generally comprise a suitable pharmaceutical excipient, diluent or carrier selected dependent on the intended route of administration.

Interferons of and for use in the present invention may be administered to a patient in need of treatment via any suitable route. The precise dose will depend upon a number of factors, including the precise nature of the interferon to be administered.

Preferred routes of administration are parentally (including subcutaneous, intramuscular, intravenous, intradermal, intrathecal and epidural), or administration via oral or nasal inhalation. Some further routes of administration include, but are not limited to oral, rectal, nasal, topical (including buccal and sublingual), vaginal, and parenteral.

In preferred embodiments, the composition is deliverable as an injectable composition, is administered orally, or is administered to the lungs as an aerosol via oral or nasal inhalation.

For administration via the oral or nasal inhalation routes, preferably the interferon composition will be in a suitable pharmaceutical formulation and may be delivered using a mechanical form including, but not restricted to an inhaler or nebuliser device.

Further, where the oral or nasal inhalation routes are used, administration is by a SPAG (small particulate aerosol generator) may be used.

For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, Lactated Ringer's injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, H. C. et al. 7^{th} Edition ISBN 0-683305-72-7 the entire disclosures of which are herein incorporated by reference.

### Pharmaceutical Compositions

As described above, the present invention extends to a pharmaceutical composition for the treatment of Influenza virus A, subtypes H5, H7 and H9 (commonly termed “avian influenza” or “bird flu”) in humans, wherein the composition comprises at least one interferon. Pharmaceutical compositions according to the present invention, and for use in accordance with the present invention may comprise, in addition to active ingredient (i.e. one or more interferon), a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be, for example parental or via oral or nasal inhalation.

The formulation may be a liquid, for example, a physiologic salt solution containing non-phosphate buffer at pH 6.8-7.6, or a lyophilised or freeze dried powder.

### Dose

The composition/interferon is preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration.

For example, in one embodiment, a suitable dose may be 1 to 10 million IU, for example 3-5 million IU three times weekly to 0.5 to 10 million, for example 2 to 8 million IU, or 4 to 6 million IU daily.

The compositions of the present invention have further utility in the identification of compounds which have efficacy in the treatment of avian influenza infection.

According to a further aspect of the present invention there is provided an assay method for determining the efficacy of a composition in the treatment of Influenza virus A, subtypes H5, H7 and H9 (commonly termed “avian influenza” or “bird flu”) in humans, wherein the composition is interferon.

In a yet further aspect of the present invention, there is provided an assay method for determining the efficacy of a candidate agent in the treatment of Influenza virus A, subtypes H5, H7 and H9 (commonly termed “avian influenza” or “bird flu”) in humans, wherein the assay method includes the steps of;
incubating virus infected cells in the presence of the candidate agent,
and determining the degree of inhibition of the cytopathic effect of the virus on the cells.

In preferred embodiments of this aspect of the invention, the method further includes the step of comparing the degree of inhibition obtained using the candidate agent with the degree of inhibition obtainable with incubation with an interferon or interferon-based product.

Preferred assays for use in the assay methods of the invention include cytopathic endpoint assays and plaque reduction assays.

Preferred features of each aspect and embodiment of the invention are as for each of the other aspects mutatis mutandis unless the context demands otherwise.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

### DETAILED DESCRIPTION OF THE INVENTION

The present invention will now be described with reference to the following examples which are provided for the purpose of illustration and are not intended to be construed as being limiting on the present invention, and further, with reference to the figures, wherein:

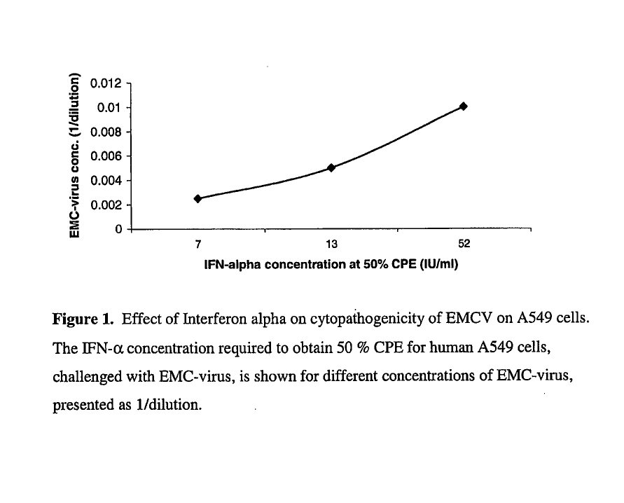

FIG. 1 shows the effect of the multiple subtype natural human alpha interferon product Multiferon™ on the cytopathogenicity of human Encephalomyocarditis virus (EMCV) on A549 cells, wherein the Multiferon™ concentration required to obtain 50% cytopathic effect (CPE) for human A549 cells challenged with EMC virus is shown for different concentrations of EMC virus;

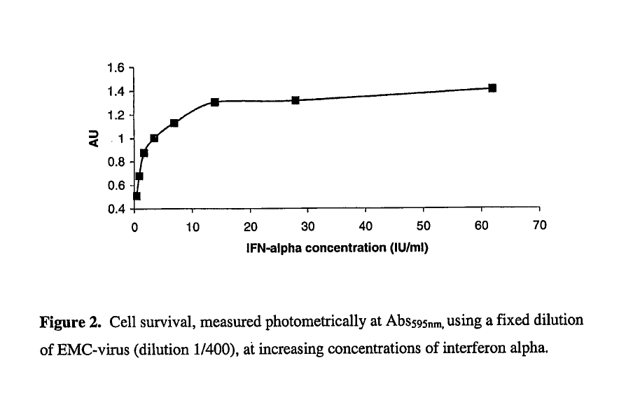

FIG. 2 shows the effect of increasing concentrations of Multiferon™ on survival of cells infected with EMCV;

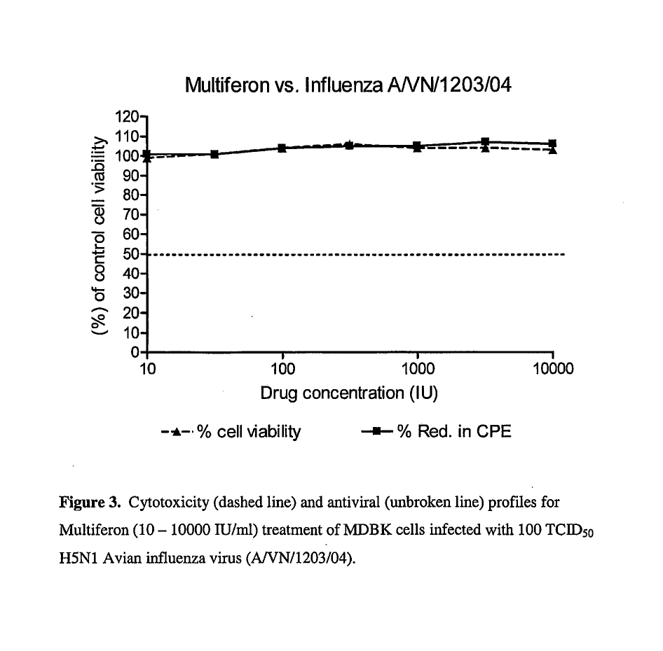

FIG. 3 shows cytotoxicity (dashed line) and antiviral (unbroken line) profiles for Multiferon (10-10000 IU/ml) treatment of MDBK cells infected with 100 TCID₅₀H5N1 Avian influenza virus (A/VN/1203/04);

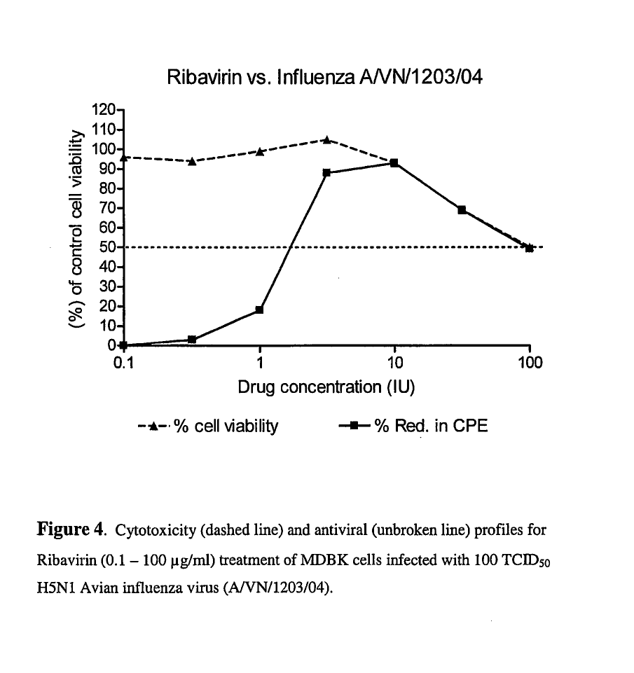

FIG. 4 shows cytotoxicity (dashed line) and antiviral (unbroken line) profiles for Ribavirin (0.1-100 μg/ml) treatment of MDBK cells infected with 100 TCID₅₀H5N1 Avian influenza virus (A/VN/1203/04);

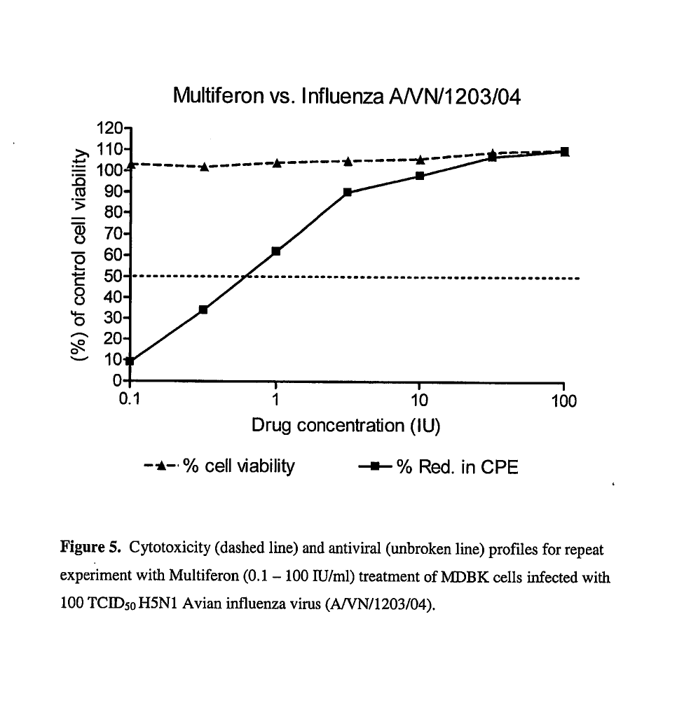

FIG. 5 shows cytotoxicity (dashed line) and antiviral (unbroken line) profiles for repeat experiment with Multiferon (0.1-100 IU/ml) treatment of MDBK cells infected with 100 TCID₅₀ H5N1 Avian influenza virus (A/VN/1203/04); and

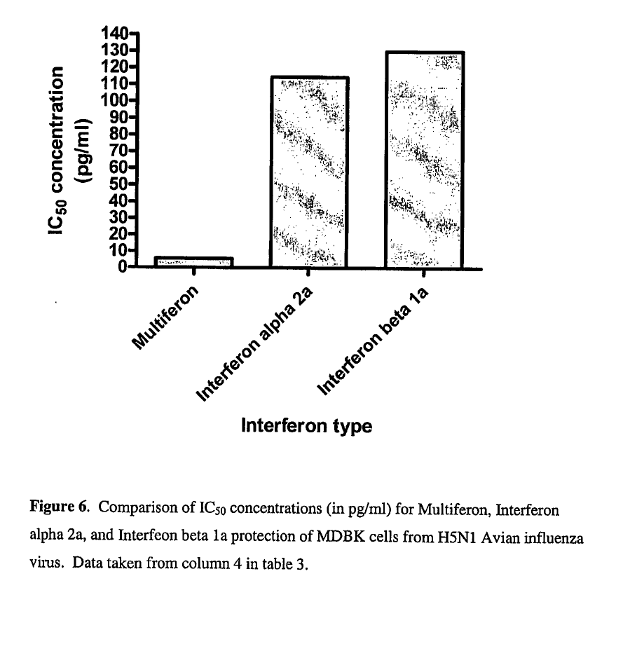

FIG. 6 shows a comparison of IC₅₀ concentrations (in pg/ml) for Multiferon, Interferon alpha 2a, and Interferon beta 1a protection of MDBK cells from H5N1 Avian influenza virus.

### EXAMPLES

### Example 1

### Anti-Viral Effect of Multi-Subtype Interferon in Human Cells

Interferons are widely known to be species specific as the target for the interferon is the infected cell rather than the virus itself.

### Cytopathic Endpoint Assay

The effect of each anti-viral treatment will be tested in quadruplicate. Briefly, 100 microlitres of serial 10-fold dilutions of each treatment was incubated with 100 microlitres of cells to give a final cell count of 20,000 cells per well in a 96-well plate. Incubation at 37° C. in 5% CO₂ was carried out overnight for the interferon preparations and for one hour for Ribavirin™. 10 microlitres of virus at a concentration of 10,000 pfu/well was then added to each test well. The plates were then incubated at 37° C. in 5% CO₂ for three days, with the plates being observed daily for cytopathic effects. The end point is the diluted concentration that inhibited the cytopathic effect in all four set-ups by 50%.

To determine cytotoxicity, 100 microlitres of serial 10-fold dilutions of each treatment was incubated with 100 microlitres of cells giving a final cell count of 20,000 cells per well in a 96-well plate, without viral challenge. The plates were then incubated at 37° C. in 5% CO₂ for three days and toxicity effects observed for using an inverted microscope.

Multiferon™ was added to human lung epithelial cells (cell line A549) prior to addition of virus. The human Encephalomyocarditis virus (EMCV) was then used to infect A549 cells and the effect of Multiferon™ on the cytopathogenicity of EMCV was determined by assessing the interferon concentration required to obtain 50% cytopathic effect (CPE) for the human A549 cells.

The results shown in FIG. 1 show the concentration of Multiferon™ needed to obtain 50% cytopathic effect in the human cells at varying viral titres. As would be expected, a higher viral concentration requires a higher effective Multiferon™ concentration.

In FIG. 2, Multiferon™ can be observed to inhibit the cytopathic effect caused by EMCV infection in a titration dependent manner.

These results show that Multiferon™ successfully inhibited cytopathic effect in EMCV-infected cells, in a titration-dependent manner.

### Example 2

### Antiviral (Anti-Influenza) and Toxicity Assays

### Materials and Methods:

Madin Darby bovine kidney (MDBK) cells were used to test the efficacy of compounds to H5N1 Avian influenza virus (H5N1; strain A/VN/1203/04). The antiviral evaluation assay examined the effects of compounds at seven half-log concentrations each. Recombinant human interferon alpha 2a and recombinant human interferon beta 1a (PBL Biomedical Laboratories, Piscataway, N.J.) as well as Ribavirin™ (MP Biomedicals, Irvine, Calif.) were included in each run as positive control compounds. Multiferon and controls were run in duplicate assays in triplicate for H5N1 as well as duplicate toxicity wells.

Subconfluent cultures of MDBK cells were plated out into 96-well plates for the analysis of cell numbers (cytotoxicity) or antiviral activity (CPE) and the next day drugs were added to the appropriate wells. One hundred 50% tissue culture infectious doses (TCID₅₀) of H5N1 or media were added to appropriate wells and cells were processed 72 hours later when the virus induced peak CPE. The effective drug concentration which reduced H5N1 CPE levels by 25% (IC₂₅), 50% (IC₅₀) and 90% (IC₉₀) were calculated by regression analysis with semi log curve fitting. H5N1 levels were assessed as relative luminescence units (RLU) using CellTiter-Glo®. The toxic concentration of drug that reduced cell numbers by 50% (TC₅₀) and 90% (TC₉₀) were calculated in the same manner. Selectivity (therapeutic) indices (SI TC/IC) at 50% (SI₅₀) and 90% (SI₉₀) were calculated.

### CellTiter-Glo® Staining for Cell Viability to Measure Cytotoxicity

At assay termination, cell viability and drug cytotoxicity were assessed using the CellTiter-Glo® Luminescent Cell Viability Assay reagent (Promega, Madison, Wis.) per the manufacturer's instructions. This reagent is a homogeneous method for determining the number of viable cells in culture based on quantitation of the ATP present, an indicator of metabolically active cells. The homogeneous assay procedure involves adding the single reagent (CellTiter-Glo® Reagent) directly to cells cultured in assay medium. The homogeneous “add-mix-measure” format results in cell lysis and generation of a “glow-type” luminescent signal (half-life generally greater than five hours) that is proportional to the amount of ATP present. The amount of ATP is directly proportional to the number of cells present in culture and readout is determined by RLU. The toxic concentration of drug that reduced cell numbers by 50% (TC₅₀) and 90% (TC₉₀) were calculated in spreadsheets by regression analysis with semi log curve fitting.

Selectivity (therapeutic) indices (SI=TC/IC) at 50% (SI₅₀) and 90% (SI₉₀) were calculated.

Multiferon™ was tested for anti-H5N1 activity using a concentration range from 10000 IU/ml down to 0.1 IU/ml. FIG. 3 details results following treatment with Multiferon at a concentration range of 10000 IU/ml down to 10 IU/ml. In this experiment, the lowest concentration of Multiferon™ used, 10 IU/ml, protected 100% of cells from H5N1 infection (unbroken line in FIG. 3) indicating that Multiferon is highly efficient at protecting cells in vitro from H5N1 infection. In contrast, 12.11 IU/ml of interferon beta 1a only protected 50% of cells from H5N1 infection (graph not shown; a summary of the results is presented in table 1 below).

TABLE 1
Summary of inhibitory concentrations and
cytotoxicity studies for Multiferon ™, interferon
beta 1a, and Ribavirin ™ against H5N1 avian influenza
virus infection of MDBK cells.

| Drug | Units | IC₂₅ | IC₅₀ | IC₉₀ | T₅₀ | SI₅₀ |
|---|---|---|---|---|---|---|
| Multiferon | IU/ml | NR | NR | NR | >10000 | — |
| Interferon | IU/ml | NR | 12.11 | 90.57 | >5000 | 412.9 |
| beta 1a | | | | | | |
| Ribavirin | μg/ml | 1.12 | 1.69 | 5.01 | 100 | 59.2 |

IC_{25/50/90} = 25/50/90% inhibitory concentration.
T₅₀ = 50% toxic concentration.
SI₅₀ = 50% selectivity index.
IU = international units/ml.
NR = not reached.

FIG. 3 (dashed line) shows that Multiferon™ was not toxic at any concentrations tested up to 10,000 IU/ml. In contrast, FIG. 4 (dashed line) shows that Ribavirin™ was toxic at concentrations above that which protected 80% of cells from H5N1 infection, i.e. it was not possible to reach full protection of cells using Ribavirin™.

FIG. 5 details results from a repeat study where the concentration range for Multiferon™ was reduced to 100 IU/ml down to 0.1 IU/ml to determine an IC₅₀ concentration for Multiferon™. Ribavirin™, interferon beta 1a and interferon alpha 2a were included in this study.

A summary of the results is presented in table 2.

TABLE 2
Summary of inhibitory concentrations and
cytotoxicity studies from repeat study, comparing
Multiferon ™, interferon alpha 2a, interferon beta 1a,
and Ribavirin ™ against H5N1 avian influenza
virus infection of MDBK cells.

| Drug | Units | IC₂₅ | IC₅₀ | IC₉₀ | T₅₀ | SI₅₀ |
|---|---|---|---|---|---|---|
| Multiferon | IU | 0.21 | 0.62 | 3.30 | >100 | 161.5 |
| Interferon | IU | 12.08 | 32.08 | 86.06 | >1000 | 31.2 |
| alpha 2a | | | | | | |
| Interferon | IU | 2.01 | 10.69 | 89.43 | >1000 | 93.6 |
| beta 1a | | | | | | |
| Ribavirin | μg/ml | 1.01 | 1.96 | T | 83.4 | 42.5 |

IC_{25/50/90} = 25/50/90% inhibitory concentration.
T₅₀ = 50% toxic concentration.
SI₅₀ = 50% selectivity index.
IU = international units/ml.
T = toxic.

Multiferon™ was demonstrated to be >17-fold stronger than interferon beta 1a and >51-fold stronger than interferon alpha 2a in protecting cells from H5N1 infection (graphs not shown, results also summarized in table 3).

Table 3 details the comparison of IC₅₀ concentrations for v to interferon alpha 2a and interferon beta 1a, in IU/ml and in pg/ml. This takes into account differences in specific activity of the products tested, demonstrating Multiferon™ is >20-fold stronger than either interferon alpha 2a or interferon beta 1a when IC₅₀ concentrations in pg/ml are compared.

TABLE 3
Comparison of IC₅₀ concentrations for
Multiferon ™ to interferon alpha 2a and interferon
beta 1a, in IU/ml and in pg/ml. Results taken from
Table 2.

| | | Fold- | | Fold- |
|---|---|---|---|---|
| | | difference | | difference |
| | | compared | | compared |
| | | to | | to |
| | IC₅₀ | IC₅₀ | IC₅₀ | IC₅₀ |
| Drug | (IU/ml) | (Multiferon) | (pg/ml) | (Multiferon) |
| Multiferon | 0.62 | — | 5.64 | — |
| Interferon | 32.08 | 51.74 | 114.64 | 20.33 |
| alpha 2a | | | | |
| Interferon | 10.69 | 17.24 | 130.01 | 23.05 |
| beta 1a | | | | |

IC₅₀ = 50% inhibitory concentration.
IU = international units/ml.

Data taken from column 4 of table 3 is graphed in FIG. 6, which shows a comparison of IC₅₀ concentrations (in pg/ml) for Multiferon™, Interferon alpha 2a, and Interferon beta 1a protection of MDBK cells from H5N1 Avian influenza virus.

### SUMMARY

It is surprising that multi-subtype forms of interferon alpha provide a robust treatment or preventative therapy against avian influenza. The present invention provides an important broad spectrum, first line of defense therapeutic product which can protect against infection with avian influenza H5N1 and likely any reassortment or variant derived therefrom. Leukocyte derived natural multi-subtype forms of interferon Alpha have no or very little tendency to give rise to neutralising antibodies, and accordingly provide a higher response rate than recombinant interferon Alpha 2 products when used therapeutically in humans. For patients who develop anti-interferon antibodies to recombinant interferon alpha 2 products, it has proven useful to follow up the treatment with a natural form of alpha interferon (Milella et al., 1995).

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

Allen G, Fantes K H. (1980) Nature 287 (5781) 408-11.
Antonelli G et al. (1991), J Interferon Res 163 882-5.
Antonelli G. (1994) Antiviral Research 24 235-244.
Antonelli G. (1997) J Interferon Cytokine Res Jul; 17 Suppl 1:S39-46.
Baigent, S. J., and McCauley, J. W. (2003) Bioessays. 25:1-15.
Cheung, C. Y. et al., (2002) The Lancet. 360:1831-1837.
De Jong, M. D. et al., (2005) NEJM 353 2667-72.
Dreitlein, W. B., et al. (2001) Clin. Ther. 23:327-355.
Easterday, B. C., et al. (1997) Influenza, p. 583-605. In B. W. Calnek, H. J. Barnes, C. W. Beard, L. R. McDougald, and Y. M. Saif (ed.). Diseases in Poultry. Iowa State University Press, Ames.
Fleming, D. M. (2001) Int. J. Clin. Pract. 55:189-195.
Farrell G C, Hepatology; 27:1121-1127.
Fish, E. N., Biochem Biophys Res Commun. 112(2) 537-546.
Foster G R, et al. 1996. J. Interferon Res. 16: 1027-1033.
Grossberg S E, Kawade Y. 1997. Biotherapy; 10(1):93-8.
Goeddel D V, et al. Nature. 290 (5801) 20-26.
Goodbourn, S. E. Y., et al. (2000) J. Gen. Virol. 81:2341-2364.
Gubareva, L. V., et al. (1998) J. Infect. Dis. 178:1257-1262.
Gubareva, L. V., et al. (2002) Antiviral-Res. 53:47-61.
Hayden, F. G., et al. (1998) J. Clin. Invest. 101:643-649.
Hilleman, M. R. (2002) Vaccine. 20:3068-3087.
Imanishi, J., et al. (1980) J. Interferon Res. 1:169-178.
Isomura, S., et al. (1982) Biken J. 25:131-137.
Kawaoka, Y., et al. (1988) Virology. 163:247-250.
Kiso, M., et al. (2004) The Lancet. 364:759-765.
Krug, R. M., et al. (2003) Virology. 309:181-189.
Lipatov, A. S., et al. (2005) J. Gen. Virol. 86:1121-1130.
Ludwig, S., et al. (2003) Trends Mol. Med. 9:46-52.
McKimm-Breschkin, J. L., et al. (1998) J. Virol. 72(3):2456-2462.
Milella M, et al. (1995) Hepatogastroenterology. Jul; 42(3):201-4.
Moscona, A. (2004) The Lancet. 364:733-734.
Roffi L, et al. (1995) Hepatology. 21(3):645-9.
Seo, S. H., et al. (2002) Nature Medicine. 8:950-954.
Seo, S. H., et al. (2004). Virus Res. 103:107-113.
Shortridge, K. F., et al. (1998). Virology. 252:331-342.
Slemons, R. D., et al. (1974). Avian Dis. 18:119-124.
Suarez, D. L., et al. (1998). J. Virol. 72:6678-6688.
Tannock, G. A., et al. (1988). Epidemiol. Infect. 101:611-621.
Taubenberger, J. K. et al. (2000). Virology. 274:241-245.
To, K. F., et al. (2001). J. Med. Virol. 63:242-246.
Weck, P. K. et al. (1981) J. Gen. Virol. 57 233-237
Von Wussow P et al. Blood 78:38-43
Von Wussow P et al. J Interferon Res. 14(4):217-9.
Weiland O et al. Scand J Infect Dis. 27: 319-324

1. A method for the treatment and/or prophylaxis of human infection with a type A Influenza virus characterised in that the virus has a hemagglutinin component of subtype H5, H7 or H9, the method comprising the steps of:
providing a composition comprising a type I interferon, and
administering a therapeutically useful amount of said composition to a subject in need of treatment.

2. A method as claimed in claim 1 wherein the subtype of the type A Influenza virus is H5N1, H9N2, H7N2, H7N3 or H7N7.

3. A method as claimed in claim 1 wherein the type I interferon is an interferon alpha subtype.

4. A method as claimed in claim 1 wherein the type I interferon is interferon beta.

5. A method as claimed in claim 1 wherein the interferon is glycosylated.

6. A method as claimed in claim 1 wherein the interferon is naturally derived.

7. A method as claimed in claim 1 wherein the interferon is comprised of at least 2 interferon alpha subtypes.

8. A method as claimed in claim 1 wherein the interferon comprises a mixture of the interferon alpha subtypes alpha 1, alpha 2, alpha 8, alpha 10, alpha 14 and alpha 21.

9. A method as claimed in claim 1 wherein the interferon alpha subtypes are present in the mixture at the following proportions by weight: alpha 1 at 37+/−9%, alpha 2 and alpha 21 at 30+/−7%, alpha 8 plus alpha 10 at 22+/−6%, and alpha 14 at 11+/−3%.

10-20. (canceled)

21. An interferon composition comprising at least interferon alpha subtypes 1, 2, 8, 10, 14 and 21 for use in the treatment of an avian influenza infection in humans.

22. The interferon composition of claim 21 wherein the interferon composition comprises interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

23. A pharmaceutical composition for the treatment of an avian influenza infection, wherein said composition comprises interferon along with a pharmaceutically acceptable excipient, carrier or diluent.

24. A pharmaceutical composition as claimed in claim 23 wherein the interferon composition may comprise interferon alpha of the subtypes 1, 2, 8, 10, 14 and 21.

25. A pharmaceutical composition as claimed in claim 23 wherein the interferon composition comprises a plurality of interferon alpha subtypes at the following percentages by weight: alpha 1 at 37+/−9%, alpha 2 and alpha 21 at 30+/−7%, alpha 8 plus alpha 10 at 22+/−6%, and alpha 14 at 11+/−3%.

26. A method for preventing or treating human infection with avian influenza, the method comprising the steps of:
providing a composition comprising a type I interferon,
administering a therapeutically useful amount of said composition to a subject in need of treatment, and
further administering a therapeutically useful amount of a suitable secondary anti-viral compound.

27. The method as claimed in claim 26 wherein the interferon is comprised of at least 2 interferon alpha subtypes.

28. The method as claimed in claim 26 wherein the interferon comprises a mixture of the interferon alpha subtypes alpha 1, alpha 2, alpha 8, alpha 10, alpha 14 and alpha 21.

29. The method as claimed in claim 26 wherein the interferon alpha subtypes are present in the mixture at the following proportions by weight: alpha 1 at 37+/−9%, alpha 2 and alpha 21 at 30+/−7%, alpha 8 plus alpha 10 at 22+/−6%, and alpha 14 at 11+/−3%.

30. The method as claimed in claim 26 wherein the secondary anti-viral compound is selected from the group consisting of ribavirin, amantadine, rimantadine, oseltamivir and zanamivir.

31-37. (canceled)
